(19) United States
(12) Patent Application Publication
(10) Pub. No.: US 2023/0293315 A1
(43) Pub. Date: Sep. 21, 2023

(54) NECK TRIAL, SURGICAL INSTRUMENT SYSTEM, AND NECK TRIAL MANUFACTURING METHOD

(71) Applicant: KYOCERA Corporation, Kyoto-shi, Kyoto (JP)

(72) Inventor: Tadaaki SHIMENO, Kakogawa-shi, Hyogo (JP)

(21) Appl. No.: 18/020,184

(22) PCT Filed: Aug. 20, 2021
(86) PCT No.: PCT/JP2021/030517

§ 371 Date: Feb. 7, 2023

(30) Foreign Application Priority Data
Aug. 25, 2020 (JP) 2020-142049

(51) Int. Cl. A61F 2/46

(52) U.S. Cl. CPC A61F 2/4684; A61F 2/4607; A61F 2002/3625

(57) ABSTRACT

A shape of a neck trial is simplified more than a known one. The neck trial includes a body portion and a fixing portion protruding from a contact surface (P) of the body portion to fix the body portion to a stem broach. The fixing portion includes a plate spring including a protruding portion that engages with a first recessed portion of the stem broach. The fixing portion and the body portion are integrally formed and can be attached to and detached from the stem broach.

### TECHNICAL FIELD

The present disclosure relates to a neck trial, a surgical instrument system, and a method for manufacturing the neck trial.

### BACKGROUND OF INVENTION

Conventionally, in a general surgical procedure for hip replacement arthroplasty, a femur is treated after installing a shell and a liner trial in an acetabulum. To fix a stem to the femur, first a hole conforming to the shape of the stem is drilled into a medullary cavity using a stem broach. While the stem broach remains placed in the femur after drilling, the stem broach is combined with a neck trial and the neck trial is combined with a ball trial, and a temporary reduction is made between the shell and the liner trial on the acetabulum. In the temporary reduction, a movable range of the hip joint, tension and dislocation resistance, and the like are confirmed, and an optimal size of a bone head ball (offset and bone head diameter) and the like are examined.

When combining the stem broach and neck trial for the temporary reduction, the neck trial may be manually removably fixed to the stem broach. When the fixation is weak, the neck trail will unintentionally fall out of the stem broach during the temporary reduction, which will make it difficult to confirm the reduction. Conversely, when the fixation is excessively strong, using a special instrument will be necessary to remove the neck trial from the stem broach, which will increase the number of procedures and impede the flow of surgery.

For example, a configuration is known in which a male neck trial and a female stem trial are combined.

Known neck trials include a neck trial in which a plate spring component is fixed with a screw or a neck trial in which a ball plunger is welded and fixed.

### SUMMARY

In one aspect of the present disclosure, a neck trial includes a body portion, and a fixing portion protruding from a contact surface of the body portion and configured to fix the body portion to a stem broach. The fixing portion includes a plate spring including a protruding portion that engages with a first recessed portion of the stem broach. The fixing portion and the body portion are integrally formed and can be attached to and detached from the stem broach.

A method for manufacturing a neck trial according to one aspect of the present disclosure is a method for manufacturing a neck trial, the neck trial including a body portion, and a fixing portion protruding from a contact surface where the stem broach and the body portion are in contact to fix the neck trial and the stem broach. The fixing portion is made of a plate spring including a protruding portion that engages with a first recessed portion formed in the stem broach, and the body portion and the fixing portion are integrally formed.

### BRIEF DESCRIPTION OF THE DRAWINGS

FIG. 1 is a cross-sectional view illustrating an artificial hip joint installed in a patient using a hip replacement arthroplasty surgical instrument according to an embodiment of the present disclosure, together with a part of the pelvis and femur.

FIG. 2 is a side view of a hip replacement arthroplasty surgical instrument.

FIG. 3 is a schematic view illustrating a configuration of the neck trial.

FIG. 4 is a side view and a partial cross-sectional view illustrating a combination of the stem broach and the neck trial.

FIG. 5 is a schematic view illustrating the neck trial according to another embodiment.

FIG. 6 is a schematic view illustrating the stem broach according to another embodiment.

### DESCRIPTION OF EMBODIMENTS

### First Embodiment

### Summary of Hip Replacement Arthroplasty Surgical Instrument 1

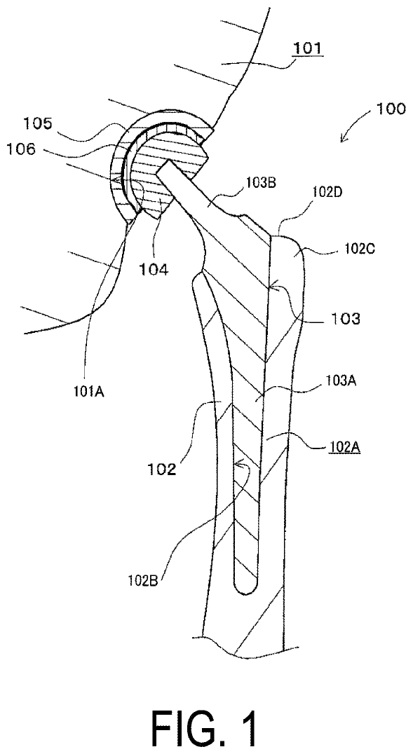

Embodiments of the present disclosure will be described below with reference to the drawings. FIG. 1 is a cross-sectional view of an artificial hip joint 100 installed in the patient using the hip replacement arthroplasty surgical instrument 1 according to an embodiment of the present disclosure, together with a part of a pelvis 101 and a femur 102. As illustrated in FIG. 1, the artificial hip joint 100 includes a stem 103, a bone head ball 104, a shell 105 and a liner 106 installed in an acetabulum 101A. Materials of the stem 103, the bone head ball 104, and the shell 105 can include, for example, metal materials such as a titanium alloy, a cobalt chromium alloy, and stainless steel, polymer materials such as polyethylene and PEEK, and ceramic materials such as alumina and zirconia, which have been authorized for use as medical devices for bioimplantation.

The stem 103 is formed in an elongated rod shape, and a middle portion of the stem 103 has a bent shape. The stem 103 includes a stem body 103A and a neck portion 103B.

The stem body 103A is inserted into a hole portion 102B formed in a medullary cavity portion 102A of the femur 102 and is fixed to the femur 102 using bone cement or the like. The stem body 103A extends along the longitudinal direction of the femur 102 and has a shape tapering from a proximal portion to a distal portion of the stem body 103A.

The neck portion 103B is provided as a portion protruding from the proximal portion of the stem body 103A. The neck portion 103B extends in a direction inclined relative to the longitudinal direction of the stem body 103A. A base end portion of the neck portion 103B is formed in a tapered shape. The bone head ball 104 is fixed to the tip of the neck portion 103B.

The bone head ball 104 is formed in a substantially spherical shape and constitutes a bone head part of the artificial hip joint 100. Note that in the present embodiment, the artificial hip joint in which the bone head ball 104 is coupled to the stem 103 is described by way of example, but need not be of this type. For example, the bone head ball 104 and the stem 103 may be integrally formed. The bone head ball 104 is received in the shell 105 via a liner 106.

The shell 105 and liner 106 are held on the pelvis 101 and cooperate with the bone head ball 104 to form a spherical joint, permitting movement of the femur 102 relative to the pelvis 101. The shell 105 is a cup-shaped member having a depression, and is fixed to the acetabulum 101A of the pelvis 101. The liner 106 is press-fitted to the shell 105.

The liner 106 is a cup-shaped member having a depression. The bone head ball 104 is slidably in contact with the inner side surface of the liner 106. With the configuration described above, the bone head ball 104 slides against the inner side surface of the liner 106 so that the femur 102 is displaceable relative to the acetabulum 101A.

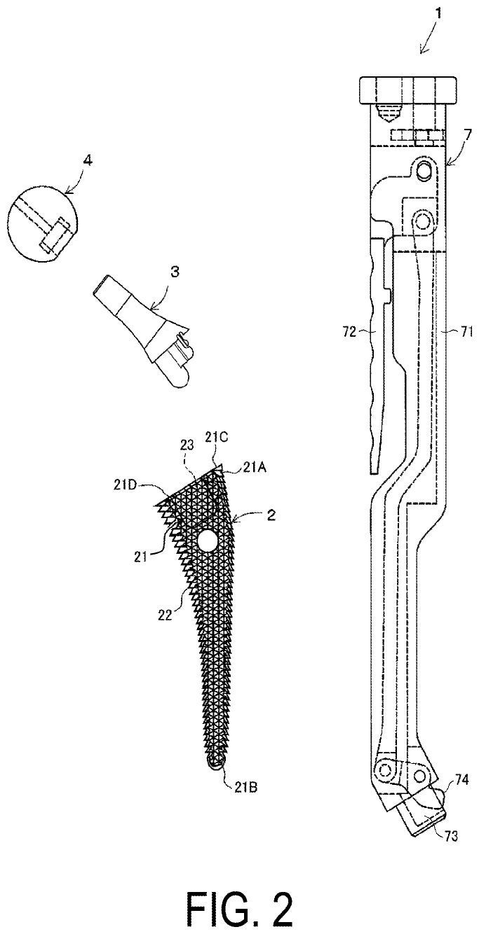

Regarding the artificial hip joint 100 having the configuration described above, the formation of the hole portion 102B, selection of the bone head ball 104, and the like are performed using the hip replacement arthroplasty surgical instrument (surgical instrument system) 1 illustrated in FIG. 2. FIG. 2 is a side view of the hip replacement arthroplasty surgical instrument 1.

As illustrated in FIG. 2, the hip replacement arthroplasty surgical instrument (hereinafter, also referred to as a surgical instrument) 1 includes a stem broach 2, a neck trial 3, a ball trial 4, and a broach handle 7.

The stem broach 2 is an osteotomy member for forming the hole portion 102B in which the stem 103 is disposed in a proximal portion 102C of the femur 102. The stem broach 2 has substantially the same contour shape as the stem 103. The stem broach 2 is formed using a metal material, for example.

The stem broach 2 includes a large number of blade portions 22 on a stem broach body 21. The stem broach body 21 includes a proximal portion 21A and a distal portion 21B. A proximal end surface 21C is formed in the proximal portion 21A. A second recessed portion 23 is formed in the proximal end surface 21C.

The second recessed portion 23 is provided as a holding portion disposed in the proximal portion 21A of the stem broach body 21, and is configured so that the neck trial 3 and the broach handle 7 can be alternatively fitted (fixed) thereto. The second recessed portion 23 has a shape recessed in a direction orthogonal to the proximal end surface 21C. The depth of the second recessed portion 23 is, for example, about 10 mm.

A first recessed portion 24 is formed on a side wall of the second recessed portion 23. The first recessed portion 24 is a depression recessed along a direction orthogonal to a side surface of the second recessed portion 23, and has a shape to which a protruding portion 34 of a fixing portion 32 of the neck trial 3 described below is fitted.

The large number of blade portions 22 are formed on an outer side surface 21D of the stem broach body 21. Note that the outer surface 21D is provided as a portion of the outer surface of the stem broach body 21 other than the proximal end surface 21C. Each of the blade portions 22 has a size of about several mm, for example. The stem broach 2 is inserted into the proximal portion 102C of the femur 102 and displaced relative to the proximal portion 102C so that the blade portion 22 cuts a bone tissue of the medullary cavity portion 102A. The neck trial 3 can be attached to and detached from the stem broach 2. The neck trial 3 is a portion provided as a neck part extending from the proximal portion 21A of the stem broach body 21 and corresponding to the neck portion 103B of the artificial hip joint 100.

A plurality of ball trials 4 having different outer diameters may be provided (in FIG. 2, only one ball trial 4 is illustrated).

The broach handle 7 is provided as an operation member for operating (moving) the stem broach 2, and can be attached to and detached from the stem broach 2. The broach handle 7 is formed in an elongated rod shape on the whole. The broach handle 7 includes a body portion 71, a lever 72, an insertion portion 73, and a hook 74.

The body portion 71 is a portion gripped by an operator. The lever 72 is operated by the operator to operate the hook 74. The insertion portion 73 is a protruding portion having a shape fitting into the second recessed portion 23 of the stem broach 2. The hook 74 is a portion protruding from the insertion portion 73 and has a shape fitting into the first recessed portion 24 of the stem broach 2. The hook 74 operates so as to be stored inside the insertion portion 73 or protrude from the insertion section 73 in accordance with the operation of the lever 72.

The operator grips the body portion 71 of the broach handle 7 and inserts the insertion portion 73 into the second recessed portion 23 of the stem broach 2 while operating the lever 72 and storing the hook 74 inside the insertion portion 73. Thereafter, the operator operates the lever 72 to cause the hook 74 to protrude from the insertion portion 73 and fit into the first recessed portion 24 of the stem broach 2. Accordingly, the broach handle 7 couples to the stem broach 2, and the stem broach 2 can be moved by moving the broach handle 7. By reversing the procedure described above, the broach handle 7 is removed from the stem broach 2.

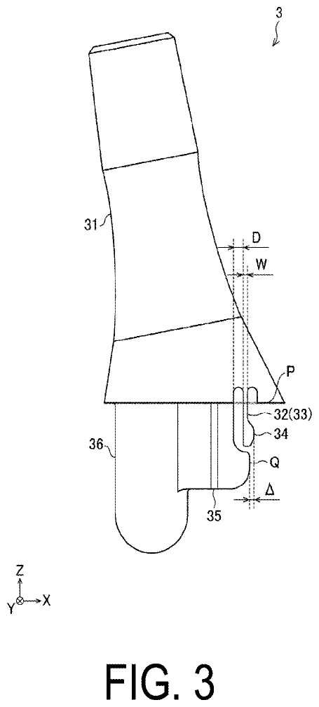

FIG. 3 is a schematic view illustrating a configuration of the neck trial 3. The neck trial 3 is a neck trial which can be attached to and detached from the stem broach 2 for hip replacement arthroplasty. As illustrated in FIG. 3, the neck trial 3 includes a body portion 31 and a fixing portion 32 protruding from a contact surface P of the body portion 31 in contact with the stem broach 2 to fix the body portion 31 to the stem broach 2. The contact surface P is a surface of the neck trial 3 that comes into contact with the stem broach 2 when the neck trial 3 is mounted on the stem broach 2. The neck trial 3 may be made of any of titanium alloy, cobalt chromium alloy, or stainless steel, as a biocompatible material.

The fixing portion 32 is a structure for suppressing detachment of the neck trial 3 mounted on the stem broach 2. The fixing portion 32 protrudes from the contact surface P of the body portion 31 toward a movement direction of the neck trial (negative direction of the Z axis in FIG. 3) when the neck trial 3 is mounted on the stem broach 2. The fixing portion 32 includes a plate spring 33 that elastically displaces (warps) in a negative X axis direction in FIG. 3. The plate spring 33 includes a protruding portion 34 that engages with the first recessed portion 24 of the stem broach 2. The protruding portion 34 protrudes from the plate spring 33 in a direction opposite to the direction in which the leaf spring 33 warps.

In the neck trial 3, the fixing portion 32 and the body portion 31 are integrally made. Here, “integrally made” means that the body portion 31 and the fixing portion 32 are inseparably and seamlessly made. For example, the body portion 31 and the fixing portion 32 may be produced from a single material (for example, a metal material) by performing machining such as cutting or grinding. Alternatively, for example, the body portion 31 and the fixing portion 32 may be produced by seamlessly welding a plurality of members. Alternatively, for example, the body portion 31 and the fixing portion 32 may be produced as a single member using a metal lamination molding technique.

The neck trial 3 may further include a protecting portion 35 protruding from the contact surface P of the body portion 31 to suppress damage to the fixing portion 32 due to contact with other objects. As illustrated in FIG. 3, the protecting portion 35 protrudes to a position farther from the body portion 31 more than the fixing portion 32 in a direction where the fixing portion 32 protrudes from the body portion 31.

As illustrated in FIG. 3, the plate spring 33 is thin in the X axis direction, and thus the strength is not high. Therefore, it is feared that the plate spring 33 of the neck trial 3 may become damaged when colliding with other instruments (e.g., stem broach 2). Here, the neck trial 3 includes the protecting portion 35. As illustrated in FIG. 3, in the fixing portion 32, only the protruding portion 34 protrudes from an outermost end surface Q of the protecting portion 35 in the X axis direction. Therefore, the fixing portion 32 is protected by the protecting portion 35, and thus the possibility of damage to the fixing portion 32 can be reduced.

The body portion 31 includes an insertion portion 36 configured to be inserted into the second recessed portion 23 of the stem broach 2, and to position the neck trial 3 relative to the stem broach 2.

A method for manufacturing the neck trial 3 will be described below. As an example, the neck trial 3 according to an embodiment may be integrally formed from the body portion 31, the fixing portion 32, and the protecting portion 35 by cutting a neck trial base material with a wire cutter. When the titanium alloy is used as a material of the neck trial 3, a projection (=hook-up allowance) A of the protruding portion 34 that achieves fixing by the fixing portion 32 may be 0.2 mm or more and 0.5 mm or less. A spring thickness W that defines a warping property of the plate spring 33 may be 0.3 mm or more and 0.5 mm or less. D, which is a warping allowance of the fixing portion 32, may be any value as long as it is greater than A.

The neck trial 3 has a simple shape made of the body portion 31, the fixing portion 32, the protecting portion 35, and the insertion portion 36, which are integrally formed. Therefore, the neck trial 3 has fewer gaps than a conventional neck trial. Since the neck trial 3 is integrally formed, there are no gaps formed as a result of combining a plurality of parts. Therefore, even when the neck trial 3 is soiled with blood or the like, there are few gaps where dirt may collect. As described above, in an embodiment, the neck trial 3 can reduce the risk of blood residue after washing after being used in surgery.

The neck trial 3 has a simple shape, and further includes the body portion 31 and the fixing portion 32 which are integrally formed. Therefore, a manufacturing process of the neck trial 3 is simplified more than a conventional one, and thus manufacturing costs are reduced. Since the plate spring 33 can be manufactured only by an NC (numerical control)-processed wire cutter, dimensions are constant, and the degree of firmness of manual attachment/removal of the neck trial 3 is stable at a constant value. Therefore, the operator can stably use the neck trial 3.

Method for Using Hip Replacement Arthroplasty Surgical Instrument 1

Described below is a method for using the hip replacement arthroplasty surgical instrument 1 including the neck trial 3 according to an embodiment. The operator prepares for installation of the artificial hip joint 100 in the patient by using the hip replacement arthroplasty surgical instrument 1. First, the patient's femoral head is removed by a surgical instrument such as a bone saw, a bone drill, a bone chisel, or the like, which is not illustrated, and further a pilot hole for inserting the stem broach 2 is formed in the medullary cavity portion 102A of the proximal portion 102C of the femur 102.

The operator couples the stem broach 2 to the broach handle 7. The operator inserts the stem broach 2 into the pilot hole of the patient's femur 102 while gripping the broach handle 7 and shaves the medullary cavity portion 102A of the proximal portion 102C. Accordingly, the hole portion 102B is formed in the femur 102, and the stem broach 2 is held in the hole portion 102B. The operator then removes the broach handle 7 from the stem broach 2 while the stem broach 2 remains in the femur 102.

Subsequently, the operator inserts and fixes the insertion portion 36 of the neck trial 3 into the second recessed portion 23 of the stem broach 2. Thereafter, the operator mounts the ball trial 4 in the neck trial 3, and performs a task of confirming a movable region of the patient's leg, and the like.

The operator will perform the same and/or similar operation for a plurality of sizes and forms (offsets) of the ball trial 4 as necessary to determine an optimal size for the patient. The operator then removes the ball trial 4 and the neck trial 3 from the stem broach 2 and proceeds to the next surgical procedure. Finally, implantation of the artificial hip joint 100 in the optimal size and form for the patient is possible.

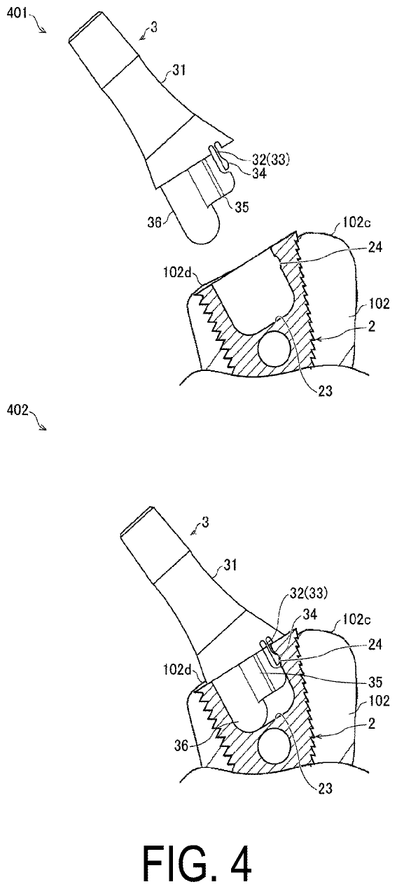

The reference sign 401 in FIG. 4 indicates a side view and a partial cross-sectional view illustrating the stem broach 2 and the neck trial 3 prior to being mounted onto the stem broach 2. The reference sign 402 in FIG. 4 indicates a side view and a partial cross-sectional view illustrating a state in which the neck trial 3 is mounted onto the stem broach 2.

In the above-described step, when the neck trial 3 is mounted in the stem broach 2, first, the plate spring 33 of the neck trial 3 is warped, as indicated by the reference sign 401 in FIG. 4. By warping the plate spring 33, the insertion portion 36 can be inserted into the second recessed portion 23 of the stem broach 2. When an end portion of the insertion portion 36 reaches an end portion of the second recessed portion 23 as a broach hole, the warp of the plate spring 33 is released, and the protruding portion 34 of the fixing portion 32 engages with the first recessed portion 24 as indicated by the reference sign 402 in FIG. 4. Accordingly, the neck trial 3 is fixed to the stem broach 2. When removing the neck trial 3, the plate spring 33 engaged with the first recessed portion 24 is warped, and thus the neck trial 3 can move inside the second recessed portion 23. In this state, removal is performed by the operator moving the insertion portion 36 of the neck trial 3 inserted into the second recessed portion 23 of the stem broach 2. In this way, the neck trial 3 can be manually attached to and detached from the stem broach 2.

### Second Embodiment

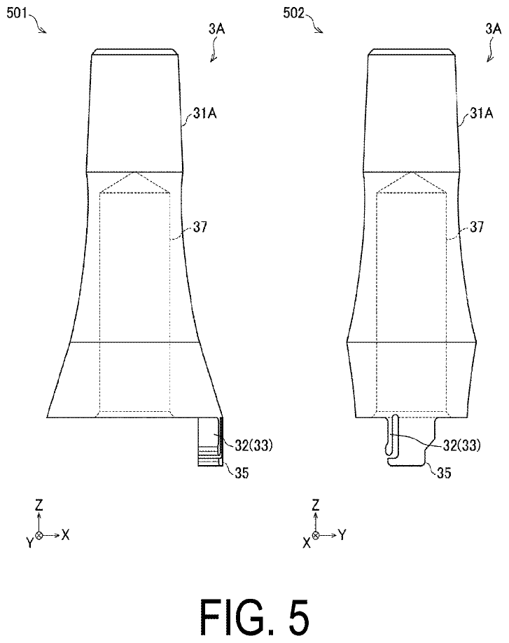

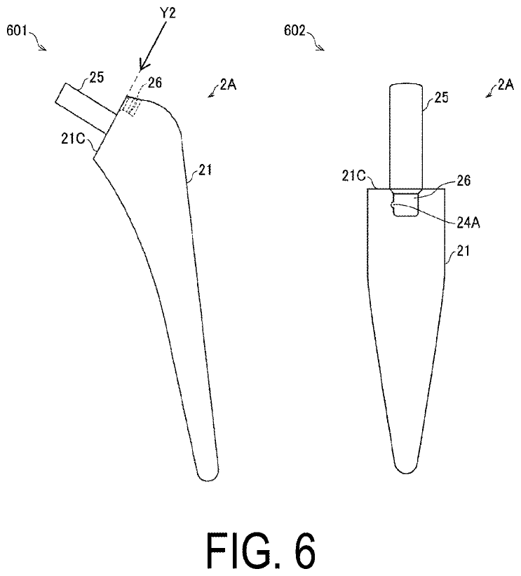

Another embodiment of the present disclosure will be described below. Note that, for convenience of description, a member having the same function as that of a member described in the embodiments described above is denoted by the same reference sign, and description thereof will not be repeated. FIG. 5 is a schematic view illustrating a neck trial 3A according to another embodiment. A diagram indicated by the reference sign 501 in FIG. 5 illustrates the neck trial 3A when viewed from a side surface, and a diagram indicated by the reference sign 502 in FIG. 5 illustrates the neck trial 3A indicated by the reference sign 501 when viewed from the X axis direction. FIG. 6 is a schematic view illustrating a stem broach 2A according to the present embodiment. A diagram indicated by the reference sign 601 in FIG. 6 illustrates the stem broach 2A when viewed from a side surface, and a diagram indicated by the reference sign 602 in FIG. 6 illustrates the stem broach 2A indicated by the reference sign 601 when viewed from a Y2 direction in the diagram.

As illustrated in FIG. 5, a body portion 31A of the neck trial 3A according to the present embodiment does not include the insertion portion 36 in the embodiment 1. Instead, the body portion 31A includes a hole portion 37 for inserting a protruding portion 25 of the stem broach 2A and positioning the neck trial 3A relative to the stem broach 2A.

As illustrated in FIG. 6, the stem broach 2A corresponding to the neck trial 3A according to the present embodiment includes the stem broach body 21, a first recessed portion 24A, the protruding portion 25, and a third recessed portion 26. The first recessed portion 24A is a depression that engages with the plate spring 33 of the neck trial 3A. The protruding portion 25 is a protruding portion protruding from the proximal end surface 21C of the stem broach 2A and inserted into the hole portion 37 of the neck trial 3A. The third recessed portion 26 is a depression formed in the proximal end surface 21C, into which the protecting portion 35 of the neck trial 3A is inserted. Note that the stem broach body 21 includes a large number of blade portions, which are not illustrated in FIG. 6.

In the present embodiment, the neck trial 3A is mounted onto the stem broach 2A. Specifically, the protruding portion 25 of the stem broach 2A inserted into the patient's femur is inserted into the hole portion 37 of the neck trial 3A. Accordingly, the neck trial 3A is positioned relative to the stem broach 2A. The fixing portion 32 and the protecting portion 35 of the neck trial 3A are inserted into the third recessed portion 26 of the stem broach 2A by warping the plate spring 33. When the protruding portion 34 of the plate spring 33 reaches the first recessed portion 24A of the third recessed portion 26, the warping of the plate spring 33 is released, and the protruding portion 34 and the first recessed portion 24A engage with each other. Accordingly, the neck trial 3A is fixed to the stem broach 2A.

Note that, in the present embodiment, the broach handle 7 whose shape fits the stem broach 2A can be used as appropriate.

Note that, in the present disclosure, the neck trial 3/3A has been explained as a member exclusively mounted onto the stem broach 2/2A, but instead of the stem broach 2/2A, the neck trial is also adaptable to a stem trial without the large number of blade portions 22 in the stem broach body 21/21C.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

### REFERENCE SIGNS

1 Hip replacement arthroplasty surgical instrument (surgical instrument system)
2, 2A Stem broach
3, 3A Neck trial
24, 24A First recessed portion
25 Protruding portion
31 Body portion
32 Fixing portion
33 Plate spring
34 Protruding portion
35 Protecting portion
36 Insertion portion
37 Hole portion
P Contact surface

1. A neck trial, comprising:
a body portion comprising a contact surface; and
a protrusion;
protruding from the contact surface and configured to fix the body portion to a stem broach that comprises a first recessed portion,
comprising a plate spring that comprises a protruding portion configured to engage with the first recessed portion; and
the fixing portion and the body portion are integrally formed and can be attached to and detached from the stem broach.

2. The neck trial according to claim 1, further comprising:
a protecting portion protruding from the contact surface.

3. The neck trial according to claim 2, wherein
the protecting portion protrudes to a position farther from the body portion than the protrusion in a direction in which the protrusion protrudes from the body portion.

4. The neck trial according to claim 2, wherein
the protecting portion is integrally formed with the protrusion and the body portion.

5. The neck trial according to claim 1, wherein
the body portion further comprises an insertion fitted to a second recessed recess of the stem broach.

6. The neck trial according to claim 5, further comprising:
a protecting portion protruding from the contact surface of the body portion to suppress damage to the protrusion due to contact with another object, wherein
the insertion portion is integrally formed with the protecting portion, the protrusion, and the body portion.

7. The neck trial according to claim 1, wherein
the body portion further comprises a hole configured to receive protruding portion of the stem broach.

8. The neck trial according to claim 1, being made of any one selected from the group consisting of titanium alloy, cobalt chromium alloy, or stainless steel, as a biocompatible material.

9. A surgical instrument system, comprising:
the neck trial according to claim 1;
a stem broach; and
a broach handle, wherein
the neck trial and the broach handle can be alternatively fixed to the stem broach.

10. The surgical instrument system according to claim 9, further comprising:
a ball trial.

11. A method for manufacturing a neck trial, the neck trial comprising:
a body portion comprising a contact surface where a stem broach and the body portion contact; and
a protrusion protruding from the contact surface and configured to fix the neck trial and the stem broach, wherein
the protrusion is made of a plate spring comprising a protruding portion configured to engage with a first recessed formed in the stem broach, and
the body portion and the protrusion are integrally formed.

12. The method for manufacturing a neck trial according to claim 11, wherein
a protecting portion protruding from the contact surface, the body portion, and the fixing portion are integrally formed.

13. The method for manufacturing a neck trial according to claim 11, wherein
a shape of the protrusion is formed by cutting with a wire cutter.

14. The method for manufacturing a neck trial according to claim 11, further comprising:
processing a single metal material by machining to make the neck trial as a single member.

15. The method for manufacturing a neck trial according to claim 11, further comprising:
producing the neck trial as a single member using a metal lamination molding technique.
